(19) United States
(12) Patent Application Publication
(10) Pub. No.: US 2016/0213286 A1
(43) Pub. Date: Jul. 28, 2016

(54) ELECTRONIC DEVICE, MONITORING AND FEEDBACK SYSTEM ON THORACOABDOMINAL MOTION AND METHOD THEREOF

(71) Applicant: National Chiao Tung University, Hsinchu City (TW)

(72) Inventors: Tzu-Chien Hsiao, Hsinchu City (TW); Pei-Shan Jhan, Taichung City (TW)

(21) Appl. No.: 14/705,942

(22) Filed: May 6, 2015

(30) Foreign Application Priority Data
Jan. 26, 2015 (TW) 104102535

(51) Int. Cl. A61B 5/113; A61B 5/00

(52) U.S. Cl. CPC A61B 5/1135; A61B 5/486; A61B 5/7203; A61B 5/7278; A61B 5/742

(57) ABSTRACT

An electronic device, a monitoring and feedback system on thoracoabdominal motion (TAM) and a method thereof are provided, where the method includes the following steps. TAM signals of a user in a natural state are measured. Next, the TAM signals are decomposed so as to extract main components thereof. Energy of the main component and the non-noise components of the abdominal motion signal are calculated to obtain the abdominal muscle contraction. Instantaneous phases of the main component of TAM signals are calculated to obtain the instantaneous coordination of TAM and the self-ability for adjusting TAM. A TAM mode of the user is evaluated in the natural state according to the abdominal muscle contraction, the instantaneous TAM coordination, and the self-ability for adjusting TAM. The user is further instructed to adjust the TAM mode to a suitable state according to a target environment mode.

### CROSS-REFERENCE TO RELATED APPLICATION

This application claims the priority benefit of Taiwan application serial no. 104102535, filed on Jan. 26, 2015. The entirety of the above-mentioned patent application is hereby incorporated by reference herein and made a part of this specification.

### BACKGROUND OF THE INVENTION

1. Field of the Invention

The invention generally relates to a monitoring technique on thoracoabdominal motion (TAM).

2. Description of Related Art

In terms of the process and mechanism of breathing, TAM is referred to as the expansion and contraction movements of the thorax and the abdomen, and its influencing factors include health statues and external environments such as gender, age, pose, breathing condition, symptom, and so forth. For respiratory function test in clinical medicine, the phase angle between the thorax motion and the abdomen motion conventionally represents the degree of thoracoabdominal asynchrony (TAA), and it serves as an evaluation index for specific respiratory functions as well as respiratory diseases and respiratory postoperative care.

However, even if the TAM is measured in a controllable environment such as a pulmonary function lab, unstable TAM of the user may cause an inaccurate phase angle in a follow-up measuring and calculating procedure. For example, measurement under unstable conditions such as user's body movement and muscle contraction may produce noises with different bandwidths. Moreover, most of the existing systems may monitor the degree of TAA considering a respiratory cycle as a unit. However, the instantaneous variation of TAA may not be monitored, and thus the instantaneous variation of the TAM during breathing may be difficult to know.

To solve the aforementioned problems that cause the results with large variations, a filtering method has been proposed to suppress noises, and yet an incorrect phase angle of TAA would be produced due to phase shifts. In terms of clinical application, despite biofeedback equipment has been developed, an effective indicator on describing instantaneous TAA, feedback and compensation on phase differences during filtering in an instantaneous coordination process of TAM, a corresponding interface for monitoring and evaluation are not yet provided.

### SUMMARY OF THE INVENTION

Accordingly, the invention is directed to an electronic device, a monitoring and feedback system on TAM and a method thereof, where a user's TAM and its instantaneous variation are able to be monitored, and the user is instructed to adjust his/her TAM mode.

The present invention is directed to a monitoring and feedback method on TAM, adapted to a system having a signal sensing device and an electronic device. The method includes the following steps. A thorax motion signal and an abdominal motion signal of a user in a natural state are measured and extracted within a predetermined time period. The thorax motion signal and the abdominal motion signal are decomposed so as to extract a main component of the thorax motion signal and a main component of the abdominal motion signal respectively. Main component energy and non-noise component energy of the abdominal motion signal are calculated, and abdominal muscle contraction is obtained accordingly. An instantaneous phase of the main component of the thorax motion signal and an instantaneous phase of the main component of the abdominal motion signal are calculated so as to obtain instantaneous coordination of the TAM and a self-ability for adjusting the TAM. A TAM mode of the user in the natural state is evaluated according to the abdominal muscle contraction, the instantaneous TAM coordination, and the self-ability for adjusting the TAM. A plurality of other environment modes are provided for selection, and the user is instructed to adjust the TAM mode to a suitable state according to a target environment mode selected from the other environment modes.

The present invention is directed to an electronic device having a screen, an input unit, a communication unit, a storage unit, and at least one processing unit, where the processing unit is coupled to the screen, the input unit, the communication unit, and the storage unit. The storage unit is configured to record a plurality of modules. The processing unit is configured to access and execute the modules recorded in the storage unit. The aforesaid modules include a receiving module, an analysis module, an evaluation module, and a feedback module. The receiving module receives a thorax motion signal and an abdominal motion signal of a user measured and extracted by a signal sensing device in a natural state through the communication unit within a predetermined time period. The analysis module decomposes the thorax motion signal and the abdominal motion signal so as to extract a main component of the thorax motion signal and a main component of the abdominal motion signal respectively, calculates main component energy and non-noise component energy of the abdominal motion signal and obtaining abdominal muscle contraction accordingly, and calculates an instantaneous phase of the main component of the thorax motion signal and an instantaneous phase of the main component of the abdominal motion signal so as to obtain instantaneous coordination of thoracoabdominal motion (TAM) and a self-ability for adjusting the TAM. The evaluation module evaluates a TAM mode of the user in the natural state according to the abdominal muscle contraction, the instantaneous TAM coordination, and the self-ability for adjusting the TAM. The feedback module provides a plurality of other environment modes for selection and instructs the user to adjust the TAM mode to a suitable state according to a target environment mode selected from the other environment modes.

The present invention is further directed to a monitoring and feedback system on TAM, where the system includes a signal sensing device and an electronic device. The signal sensing device is configured to measure and extract a thorax motion signal and an abdominal motion signal of a user in a natural state within a predetermined time period. The electronic device is configured to receive the thorax motion signal and the abdominal motion signal from the signal sensing device, decompose the thorax motion signal and the abdominal motion signal so as to extract a main component of the thorax motion signal and a main component of the abdominal motion signal respectively. The electronic device is configured to further calculate main component energy and non-noise component energy of the abdominal motion signal and obtaining abdominal muscle contraction accordingly, calculate an instantaneous phase of the main component of the thorax motion signal and an instantaneous phase of the main component of the abdominal motion signal so as to obtain instantaneous coordination of the TAM and a self-ability for adjusting the TAM, and evaluate a TAM mode of the user in the natural state according to the abdominal muscle contraction, the instantaneous TAM coordination, and the self-ability for adjusting the TAM. The electronic device is also configured to provide a plurality of other environment modes for selection and instruct the user to adjust the TAM mode to a suitable state according to a target environment mode selected from the other environment modes.

In summary, in the electronic device, a monitoring and feedback system on TAM and a method thereof proposed in the invention, a thorax motion signal and an abdominal motion signal of a user in a natural state are decomposed to extract main components of the two signals and to further evaluate abdominal muscle contraction. Instantaneous phases of the main component of the thorax motion signal and the abdominal motion are calculated so as to evaluate instantaneous coordination of TAM and a self-ability for adjusting TAM. Based on the aforesaid evaluated result, the user would be instructed to self-adjust his/her breathing according to the selected target environment mode.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 illustrates a block diagram of a monitoring and feedback system on TAM according to an embodiment of the invention.

FIG. 2 illustrates a flowchart of a monitor and feedback method on TAM according to an embodiment of the invention.

FIG. 3 illustrates a functional block diagram of a monitor and feedback method on TAM according to an embodiment of the invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts. In addition, the specifications and the like shown in the drawing figures are intended to be illustrative, and not restrictive. Therefore, specific structural and functional detail disclosed herein are not to be interpreted as limiting, but merely as a representative basis for teaching one skilled in the art to variously employ the present invention.

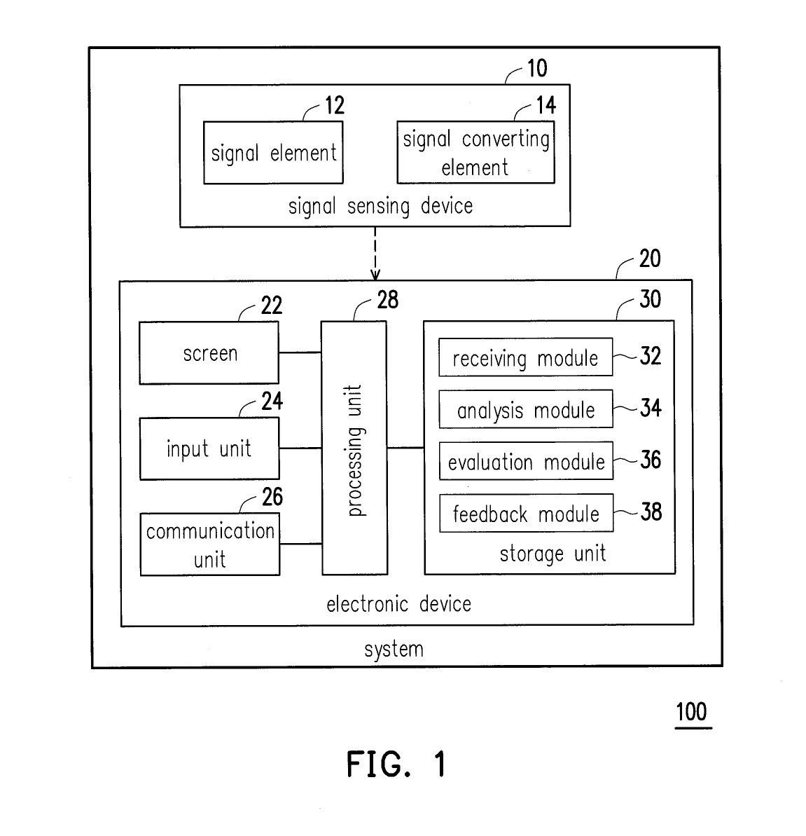

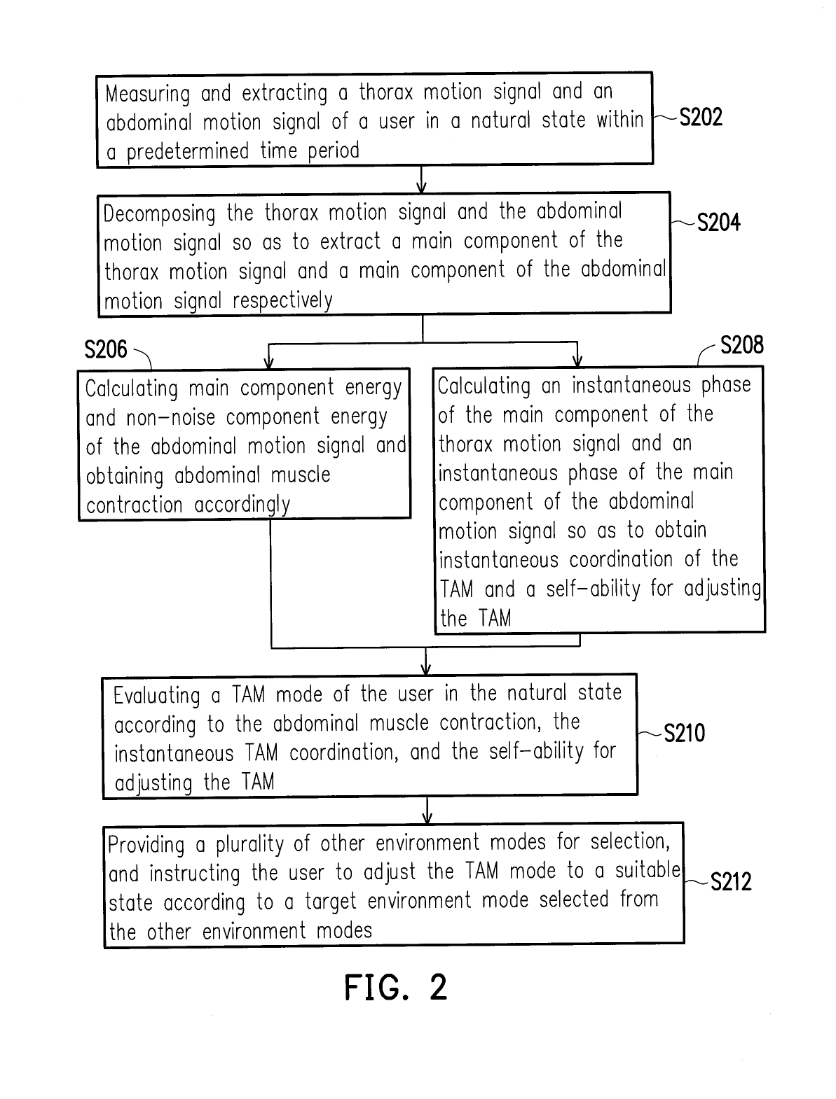

FIG. 1 illustrates a block diagram of a monitoring and feedback system on TAM according to an embodiment of the invention. It should, however, be noted that this is merely an illustrative example and the invention is not limited in this regard. All components of the monitoring and feedback system on TAM and their configurations are first introduced in FIG. 1. The detailed functionalities of the components are disclosed along with FIG. 2.

Referring to FIG. 1, a system 100 includes a signal sensing device 10 and an electronic device 20.

The signal sensing device 10 includes a sensing element 12 and a signal converting element 14, where the signal converting element 14 is coupled to the sensing element 12. The sensing element 12 may be, for example, a piezoelectric (PZT) element configured to be placed on human skin for continuously sensing signals from thorax and abdomen areas. The signal converting element 14 may be, for example, an analog-to-digital converter (ADC) configured to convert signals received by the signal sensing device 10 to digital signals which are able to be processed by the electronic device 20. The signal sensing device 10 may be implemented as a non-implanted sensor such as a sensing suit, a respiratory belt, an adhesive sensing marker, and so forth. The invention is not limited herein.

The electronic device 20 includes a screen 22, an input unit 24, a communication unit 26, a processing unit 28, and a storage unit 30. In the present embodiment, the electronic device 20 may be, for example, a smart phone, a personal digital assistant (PDA), a tabular computer, a laptop computer, a desktop computer, a digital multimedia device, an electronic entertainment device, an on-vehicle electronic display, and so forth. The invention is not limited herein.

The screen 22 is configured to display a frame output by the electronic device 20 for the user. In the present embodiment, the screen 22 may be a liquid crystal display (LCD), a light-emitting diode (LED) display, a field emission display (FED) or other types of displays. The input unit 24 is configured to provide an input feature for the user to operate the electronic device 20 and may be an input device of the electronic device 20 such as an external or built-in keyboard, mouse, stylus pen, touch panel, trackball, and so forth. In an embodiment, the screen 22 and the input unit 24 may be integrated as a touch screen (e.g. a resistive touch screen or a capacitive touch screen) which is configured to detect a touch operation performed thereon.

The communication unit 26 is configured to receive signals from the signal sensing device 10 through wireless transmission or wired transmission. For example, the communication unit 26 may support short-range communication such as infrared, Bluetooth, and near field communication (NFC) as well as wireless internet access such as WiMAX, Wi-Fi, 2G, 3G, or 4G. However, the invention is not limited thereto.

The processing unit 28 may be, for example, a central processing unit (CPU) or other programmable devices for general purpose or special purpose such as a microprocessor and a digital signal processor (DSP), a programmable controller, an application specific integrated circuit (ASIC), a programmable logic device (PLD) or other similar devices or a combination of the aforesaid devices. The processing unit 28 is coupled to the screen 22, the input unit 24, the communication unit 26, and the storage unit 30 and configured to perform the proposed monitor and feedback method on TAM.

The storage unit 30 may be one or a combination of a stationary or mobile random access memory (RAM), a read-only memory (ROM), a flash memory, a hard drive or other similar devices. The storage unit 30 is configured to record a plurality of modules executable by the processing unit 28. The modules include a receiving module 32, an analysis module 34, an evaluation module 36, and a feedback module 38, where the modules may be loaded into the processing unit 28 for performing the proposed monitor and feedback method on TAM.

FIG. 2 illustrates a flowchart of a monitor and feedback method on TAM according to an embodiment of the invention. The method in the present embodiment may be implemented by the system 100 in FIG. 1. For example, while the user is commuting, walking, watching entertaining media, sitting in an office or at home, he/she is able to self-adjust his/her breathing in an economical and convenient fashion. Detailed steps of the proposed method would be illustrated along with the components of the system 100.

Referring to both FIG. 1 and FIG. 2, the signal sensing device 10 measures and extracts a thorax motion signal and an abdominal motion signal of the user in a natural state within a predetermined time period (Step S202). To be specific, in the current step, the user may wear the signal sensing device 10 when the human body is in the natural state, and then the sensing element 12 of the signal sensing device 20 is able to sense the signals generated from the thorax and abdomen areas continuously during his/her breathing. The signal converting element 14 may convert such continuous signals to a thorax motion signal and an abdominal motion signal in a digital format. In the present embodiment, the predetermined time period may be, for example, five minutes. After the receiving module 32 of the electronic device 20 receives the thorax motion signal and the abdominal motion signal from the signal sensing device 10 via the communication unit 26, it may synchronously display the received signals on the screen 22.

Next, the analysis module 34 of the electronic device 20 decomposes the thorax motion signal and the abdominal motion signal so as to extract a main component of the thorax motion signal and a main component of the abdominal motion signal respectively (Step S204). To be specific, the analysis module 34 may perform data decomposition on the thorax motion signal and the abdominal motion signal to extract main components of the breathing motion. Thus, all uncertainties caused by noises may be reduced, and a follow-up evaluation may be more accurate.

In the present embodiment, since the thorax motion signal and the abdominal motion signal are both non-linear and non-stationary, the analysis module 34 may decompose the thorax motion signal and the abdominal motion signal respectively into a plurality of intrinsic mode functions (IMF) corresponding to different characteristic time scales by using a complementary ensemble empirical mode decomposition (CEEMD) method. Next, the analysis module 34 may extract the main components of the thorax motion signal and the abdominal motion signal from the intrinsic mode functions corresponding to the thorax motion signal and the abdominal motion signal respectively.

After the analysis module 34 extracts the main components of the thorax motion signal and the abdominal motion signal respectively, it would calculate evaluation indicators. In the present embodiment, the analysis module 34 calculates main component energy and non-noise component energy of the abdominal motion signal and obtains abdominal muscle contraction accordingly (Step S206), and further calculates an instantaneous phase of the main component of the thorax motion signal and an instantaneous phase of the main component of the abdominal motion signal so as to obtain instantaneous coordination of the TAM and a self-ability for adjusting the TAM (Step S208). It should be noted that, the execution order of Step S206 and Step 208 are not limited in the present embodiment.

In Step S206, the analysis module 34 may observe the abdominal muscle contraction based on the proportion of the main component energy in the non-noise component energy. The analysis module 34 may first obtain each component of the abdominal motion signal from the intrinsic mode functions corresponding to the abdominal motion signal obtained in Step S204. The analysis module 34 may then calculate energy and an average period of each of the components of the abdominal motion signal, and thereby eliminate noise components from the components so as to obtain the non-noise components. Next, the analysis module 34 may calculate the proportion of the main component energy in the non-noise component energy and thereby obtain the abdominal muscle contraction.

In Step S208, the analysis module 34 may calculate the instantaneous phase of the main component of the thorax motion signal and the instantaneous phase of the main component of the abdominal motion signal by using, for example, a normalized direct quadrature (NDQ) method. The analysis module 34 may further calculate instantaneous phase synchronization (IPS) between the two signals by setting the instantaneous phase of the main component of the thorax motion signal as a reference value, and thereby obtain a plurality of detailed indicators. In the present embodiment, the detailed indicators may be a full width at half maximum (FWHM) of a distribution curve of the IPS, a vibration amplitude and a vibration amplitude of each respiratory cycle and may represent the instantaneous coordination of the TAM and the self-ability for adjusting the TAM.

Next, the evaluation module 34 of the electronic device 20 evaluates a TAM mode of the user in the natural state according to the abdominal muscle contraction, the instantaneous TAM coordination, and the self-ability for adjusting the TAM (Step S210). To be specific, the evaluation module 34 may evaluate the TAM mode of the user in the natural state according to data of parameters associated with the TAM. In the present embodiment, the evaluation module 34 may set the abdominal muscle contraction, the FWHM of the IPS distribution curve of the instantaneous coordination associated with the TAM, and the vibration amplitude and the vibration amplitude of each respiratory cycle associated with the self-ability for adjusting the TAM as four independent variables to evaluate the TAM mode of the user in the natural state by using a multivariate analysis method. Moreover, since the TAM may be affected differently by physical and psychological states and activities, the storage unit 30 may further include a database to store a huge amount of reference data in an embodiment.

After the evaluation module 36 evaluates the TAM mode of the user in the natural state, the feedback module 38 provides a plurality of other environment modes for selection and instructs the user to adjust the TAM mode to a suitable state according to a target environment mode selected from the other environment modes 9Step S212). To be specific, since the evaluation module 36 may only evaluate the TAM mode of the user in the natural state, the feedback module 38 may provide a plurality of different environment modes for the user to select. The aforesaid other environment modes may be, for example, a jogging mode, an hiking mode, a walking mode, an on-vehicle mode, and so forth. In the present embodiment, the environment modes may be display on the screen 22 for selection, and the user may be able to select the current environment mode (i.e. the aforesaid target environment mode) through the input unit 24. After the feedback module 38 receives a selection signal corresponding to the target environment mode through the input unit 24, it may provide an auxiliary instruction for the user to adjust the TAM mode according to the target environment mode. For example, the feedback module 38 may concurrently monitor the TAM mode of the user and instruct the user to adjust his/her breathing to the suitable state so as to assist the user to self-adjust the TAM mode. Such auxiliary instruction may be, for example a text instruction, a graphic instruction, or a voice instruction, and yet the invention is not limited thereto.

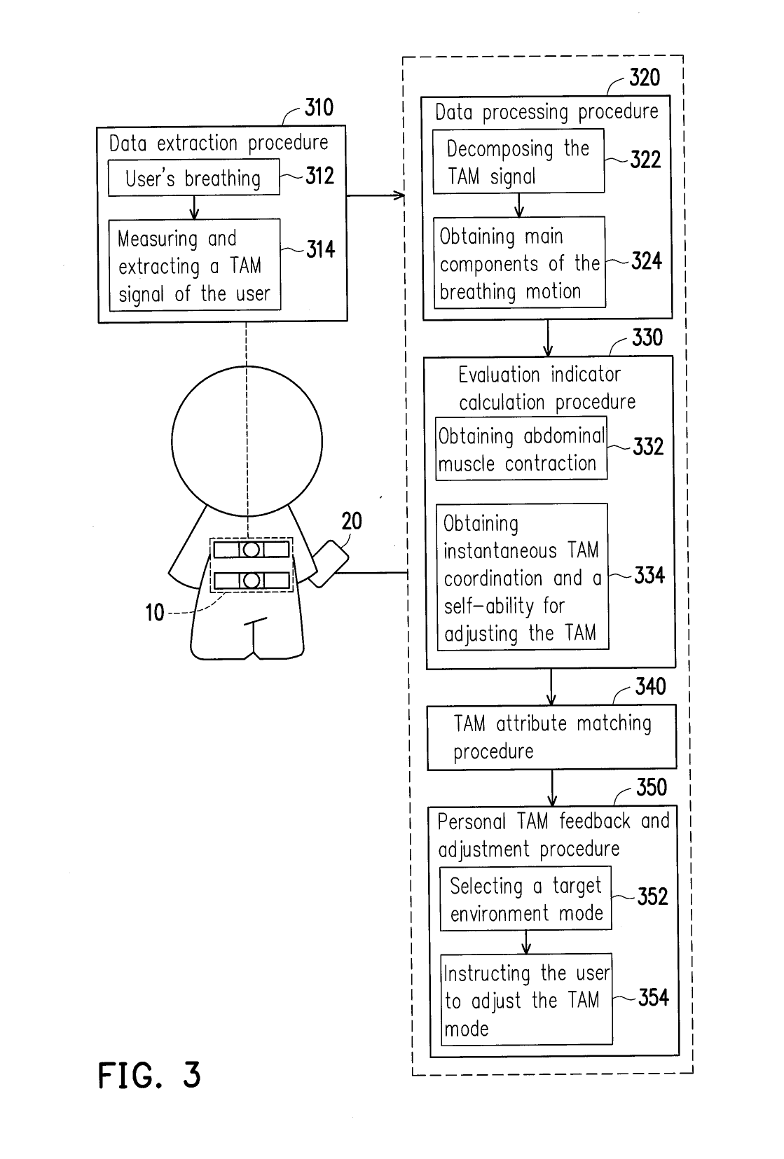

The aforesaid monitoring and feedback method on TAM may be summarized by FIG. 3 in terms of a function block diagram according to an embodiment of the invention. Referring to FIG. 3, the signal sensing device 10 first perform a data extraction procedure 310 to measure and extract a TAM signal 314 of the user during his/her breathing 312. Next, the electronic device 20 performs a data processing procedure 320 to decompose the TAM signal 322 and obtain main components of the breathing motion 324. Thereafter, the electronic device 20 performs an evaluation indicator calculation procedure 330 to obtain abdominal muscle contraction 332 as well as instantaneous TAM coordination and a self-ability for adjusting the TAM 334. The electronic device 20 then performs a TAM attribute matching procedure 340 to evaluate a TAM mode of the user in a natural state by using the evaluation indicator. Lastly, the electronic device 20 performs a personal TAM feedback and adjustment procedure 350. The user is allowed to select a target environment mode 352, and the electronic device 20 would instruct the user to adjust the TAM mode to a suitable state 354 according to the target environment mode.

In summary, in the electronic device, a monitoring and feedback system on TAM and a method thereof proposed in the invention, a thorax motion signal and an abdominal motion signal of a user in a natural state are decomposed to extract main components of his/her breathing motion and to further evaluate abdominal muscle contraction. Instantaneous phases of the main component of the thorax motion signal and the abdominal motion are calculated to obtain IPS therebetween, and instantaneous coordination of TAM and a self-ability for adjusting TAM are evaluated. Based on the aforesaid evaluated result, the user may be instructed to self-adjust his/her breathing according to the selected target environment mode. Accordingly, in the invention, the user's TAM and its instantaneous variation are able to be monitored in different measuring environments, and the instruction to adjust the TAM mode to a suitable state is feedback to the user so that he/she is able to self-adjust his/her breathing anytime anywhere.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A monitoring and feedback method on thoracoabdominal motion (TAM), adapted to a system having a signal sensing device and an electronic device, comprising:
measuring and extracting a thorax motion signal and an abdominal motion signal of a user in a natural state within a predetermined time period;
decomposing the thorax motion signal and the abdominal motion signal so as to extract a main component of the thorax motion signal and a main component of the abdominal motion signal respectively;
calculating main component energy and non-noise component energy of the abdominal motion signal and obtaining abdominal muscle contraction accordingly;
calculating an instantaneous phase of the main component of the thorax motion signal and an instantaneous phase of the main component of the abdominal motion signal so as to obtain instantaneous coordination of the TAM and a self-ability for adjusting the TAM;
evaluating a TAM mode of the user in the natural state according to the abdominal muscle contraction, the instantaneous TAM coordination, and the self-ability for adjusting the TAM; and
providing a plurality of other environment modes for selection, and instructing the user to adjust the TAM mode to a suitable state according to a target environment mode selected from the other environment modes.

2. The method according to claim 1, wherein the step of decomposing the thorax motion signal and the abdominal motion signal so as to extract the main component of the thorax motion signal and the main component of the abdominal motion signal respectively comprises:
decomposing the thorax motion signal and the abdominal motion signal respectively into a plurality of intrinsic mode functions with different characteristic time scales by using a complementary ensemble empirical mode decomposition method;
extracting the main component of the thorax motion signal from the intrinsic mode functions corresponding to the thorax motion signal; and
extracting the main component of the abdominal motion signal from the intrinsic mode functions corresponding to the abdominal motion signal.

3. The method according to claim 2, wherein the step of calculating the main component energy and the non-noise component energy of the abdominal motion signal and obtaining abdominal muscle contraction accordingly comprises:
obtaining each component of the abdominal motion signal from the intrinsic mode functions corresponding to the abdominal motion signal;
calculating energy and an average period of each of the components of the abdominal motion signal;
obtaining a plurality of noise components according to the energy and the average period of each of the components;
eliminating the noise components from the components so as to obtain the non-noise component energy; and
calculating a proportion of the main component energy in the non-noise component energy so as to obtain the abdominal muscle contraction.

4. The method according to claim 1, wherein the step of calculating the instantaneous phase of the main component of the thorax motion signal and the instantaneous phase of the main component of the abdominal motion signal so as to obtain the instantaneous coordination of the TAM and the self-ability for adjusting the TAM comprises:
calculating the instantaneous phase of the main component of the thorax motion signal and the instantaneous phase of the main component of the abdominal motion signal by using a normalized direct quadrature method;
calculating instantaneous phase synchronization by setting the instantaneous phase of the main component of the thorax motion signal as a reference value; and
obtaining a plurality of detailed indicators by using the instantaneous phase synchronization and obtaining the instantaneous coordination of the TAM and the self-ability for adjusting the TAM accordingly.

5. The method according to claim 4, wherein the detailed indicators comprise a full width at half maximum of a distribution curve of the instantaneous phase synchronization, a vibration amplitude and a vibration amplitude of each respiratory cycle in the instantaneous phase synchronization.

6. The method according to claim 1, wherein the step of evaluating the TAM mode of the user in the natural state according to the abdominal muscle contraction, the instantaneous TAM coordination, and the self-ability for adjusting the TAM comprises:
setting the abdominal muscle contraction, the instantaneous TAM coordination, and the self-ability for adjusting the TAM as independent variables to evaluate the TAM mode of the user in the natural state according to the abdominal muscle contraction by using a multivariate analysis method.

7. The method according to claim 1, wherein the step of providing the other environment modes for selection, and instructing the user to adjust the TAM mode to the suitable state according to the target environment mode selected from the other environment modes comprises:
receiving a selection signal to set the other environment mode corresponding to the selection signal as the target environment mode; and
instructing the user to adjust the TAM mode to the suitable state meeting the target environment mode.

8. An electronic device comprising:
a screen;
an input unit;
a communication unit;
a storage unit, recording a plurality of modules; and
a processing unit, coupled to the screen, the input unit, the communication unit, and the storage unit, and accessing and executing the modules recorded in the storage unit, wherein the modules comprise:
a receiving module, receiving a thorax motion signal and an abdominal motion signal of a user measured and extracted by a signal sensing device in a natural state through the communication unit within a predetermined time period;
an analysis module, decomposing the thorax motion signal and the abdominal motion signal so as to extract a main component of the thorax motion signal and a main component of the abdominal motion signal respectively, calculating main component energy and non-noise component energy of the abdominal motion signal and obtaining abdominal muscle contraction accordingly, and calculating an instantaneous phase of the main component of the thorax motion signal and an instantaneous phase of the main component of the abdominal motion signal so as to obtain instantaneous coordination of thoracoabdominal motion (TAM) and a self-ability for adjusting the TAM;
an evaluation module, evaluating a TAM mode of the user in the natural state according to the abdominal muscle contraction, the instantaneous TAM coordination, and the self-ability for adjusting the TAM; and
a feedback module, providing a plurality of other environment modes for selection, and instructing the user to adjust the TAM mode to a suitable state according to a target environment mode selected from the other environment modes.

9. The electronic device according to claim 8, wherein the analysis module decomposes the thorax motion signal and the abdominal motion signal respectively into a plurality of intrinsic mode functions with different characteristic time scales by using a complementary ensemble empirical mode decomposition method, extracts the main component of the thorax motion signal from the intrinsic mode functions corresponding to the thorax motion signal, and extracts the main component of the abdominal motion signal from the intrinsic mode functions corresponding to the abdominal motion signal.

10. The electronic device according to claim 9, wherein the analysis module obtains each component of the abdominal motion signal from the intrinsic mode functions corresponding to the abdominal motion signal, calculates energy and an average period of each of the components of the abdominal motion signal, obtains a plurality of noise components according to the energy and the average period of each of the components, eliminates the noise components from the components so as to obtain the non-noise component energy, and calculates a proportion of the main component energy in the non-noise component energy so as to obtain the abdominal muscle contraction.

11. The electronic device according to claim 8, wherein the analysis module calculates the instantaneous phase of the main component of the thorax motion signal and the instantaneous phase of the main component of the abdominal motion signal by using a normalized direct quadrature method, calculates instantaneous phase synchronization by setting the instantaneous phase of the main component of the thorax motion signal as a reference value, and obtains a plurality of detailed indicators by using the instantaneous phase synchronization and obtaining the instantaneous coordination of the TAM and the self-ability for adjusting the TAM accordingly.

12. The electronic device according to claim 8, wherein the detailed indicators comprise a full width at half maximum of a distribution curve of the instantaneous phase synchronization, a vibration amplitude and a vibration amplitude of each respiratory cycle in the instantaneous phase synchronization.

13. The electronic device according to claim 8, wherein the evaluation module sets the abdominal muscle contraction, the instantaneous TAM coordination, and the self-ability for adjusting the TAM as independent variables to evaluate the TAM mode of the user in the natural state according to the abdominal muscle contraction by using a multivariate analysis method.

14. The electronic device according to claim 8, wherein the feedback module receives a selection signal to set the other environment mode corresponding to the selection signal as the target environment mode and instructs the user to adjust the TAM mode to the suitable state meeting the target environment mode.

15. A monitoring and feedback system on thoracoabdominal motion (TAM) comprising:
a signal sensing device, measuring and extracting a thorax motion signal and an abdominal motion signal of a user in a natural state within a predetermined time period;
an electronic device, receiving the thorax motion signal and the abdominal motion signal from the signal sensing device, decomposing the thorax motion signal and the abdominal motion signal so as to extract a main component of the thorax motion signal and a main component of the abdominal motion signal respectively, calculating main component energy and non-noise component energy of the abdominal motion signal and obtaining abdominal muscle contraction accordingly, calculating an instantaneous phase of the main component of the thorax motion signal and an instantaneous phase of the main component of the abdominal motion signal so as to obtain instantaneous coordination of the TAM and a self-ability for adjusting the TAM, evaluating a TAM mode of the user in the natural state according to the abdominal muscle contraction, the instantaneous TAM coordination, and the self-ability for adjusting the TAM, and providing a plurality of other environment modes for selection, and instructing the user to adjust the TAM mode to a suitable state according to a target environment mode selected from the other environment modes.

16. The system according to claim 15, wherein the signal sensing device comprises:
a sensing element, sensing signals generated from thorax and abdomen areas of a user during the user's breathing; and
a signal converting element, coupled to the sensing element, and converting the signals generated from the thorax and abdomen areas to the thorax motion signal and the abdominal motion signal able to be processed by the electronic device.

17. The system according to claim 16, wherein the electronic device receives the thorax motion signal and the abdominal motion signal from the signal sensing device through wireless transmission or wired transmission.

18. The system according to claim 16, wherein the electronic device comprises:
a screen, displaying the thorax motion signal and the abdominal motion signal, and instructing the user to adjust the TAM mode.
